(19) United States
(12) Patent Application Publication
(10) Pub. No.: US 2017/0080185 A1
(43) Pub. Date: Mar. 23, 2017

(54) SYSTEM AND PROCEDURE FOR PLACING A MEDICAL DEVICE PROXIMATE AN OSTIAL LESION USING A CATHETER ASSEMBLY

(71) Applicant: KASSAB KUGHN ENDOVASCULAR DEVICES LLC, Farmington, MI (US)

(72) Inventors: Elias H. KASSAB, Grosse Pointe Shores, MI (US); Andrew BLACK, Johnsburg, IL (US); Mark E. ZYZELEWSKI, Kalamazoo, MI (US)

(21) Appl. No.: 15/277,175

(22) Filed: Sep. 27, 2016

Related U.S. Application Data
(63) Continuation-in-part of application No. 12/562,713, filed on Sep. 18, 2009, now abandoned.

(51) Int. Cl. A61M 25/00; A61F 2/962; A61M 25/01

(52) U.S. Cl. CPC A61M 25/0082; A61M 25/0147; A61M 25/0108; A61F 2/962; A61M 25/0136; A61M 2025/0096

(57) ABSTRACT

A catheter system and method 10 for placing a medical device or medicine proximate an ostium. The catheter system 10 has a guide catheter 14 and a sheath 22 with an internal lumen 24 surrounds the guide catheter 14. One or more connecting components 34 extend axially form an actuating mechanism 38. An ostial locating member 42 extends from the far end 22 of the catheter 14. The ostial locating member 42 has flexible, radially extending struts 44 lie in an ostial plane 48 when they are advanced against the ostium after the expandable distal legs 70 have been expanded radially outward beyond the distal end 18 of the guiding catheter 14.

### CROSS-REFERENCE TO RELATED APPLICATIONS

This application is a continuation in part of co-pending U.S. Ser. No. 12/562,713 filed on Sep. 18, 2009, which is incorporated herein by reference.

### BACKGROUND OF THE INVENTION

(1) Field of the Invention

The invention relates to a catheter assembly for positioning a medical device, such as a stent, at or near the site of an ostial lesion.

(2) Description of Related Art

In medicine, a “stent” is a man-made tube that is inserted into a natural passage/conduit in the body to prevent, or counteract a disease-induced, localized flow constriction. http://en.wikipedia/org/wiki/stent#peripheralvascular. As used herein, the term “stent” includes for example an endovascular, cylindrical, mesh-like but dilatable structure which is inserted into various atherosclerotic arteries to maintain the patency of the vessel. Usually the diseased artery has been dilated to remove the blockage with an angioplasty balloon, which has been introduced percutaneously within a tube or catheter inserted into the right femoral artery, for instance, and guided to the diseased arterial site. Conventionally, after the stent is inserted, it is left in place to maintain the opening in the previously blocked artery. Predictable, repeatable and precise placement of the stent may be challenging using conventional techniques.

During interventional vascular procedures, situations arise where a stent must be placed at the ostium of an artery. The ostium is the opening (or mouth) of an artery into another artery, such as the aorta. Ostial lesions involve for example the junction of the aorta and the origin of the right coronary artery, left main stem or a saphenous vein graft. Such lesions are called “aorto-ostial”. Other ostial lesions occur at the origin of the left anterior descending artery of the left circumflex artery arising from the bifurcation of the distal left main stem. Still other lesions may recur in the iliac and/or femoropopliteal arteries. As used herein, the term “ostial lesions” is used to describe these types of lesions. Inaccurate placement of the stent at the ostium of an artery can result in multiple risks to the patient, repeated procedures and related costs.

The location of ostial lesions makes them difficult to treat by percutaneous coronary intervention. Accurate stent positioning to avoid stent protrusion into the left main stem, for example, remains challenging. Aziz & Ramsdale, “SIROLIMUS—ELUTING STENTS FOR THE TREATMENT OF ATHEROSCLEROTIC OSTIAL LESIONS”, 17 Jnl. Invasive Cardiology, Jan. 3, 2005. If the stent is deployed too proximal to the ostium, the stent could protrude into the aorta. In such cases, future re-engagement of the vessel could be difficult. The only option for a patient who has restenosis of an artery could be bypass surgery. If the stent is deployed too distal to the ostium, additional stents may be required to fully open the lesion. This will result in increased cost of the procedure, and a higher risk for thrombosis and restenosis of the stented area.

There are devices that aid in the placement of stents at the ostium of vessels. One such device is the “Ostial Pro™” from Ostial Solutions, LLC. The Ostial Pro is a nitinol device that is positioned within a guiding catheter. http://www3.interscience.wiley.com/journal/117916410/abstract. It has distal, self-expanding legs that are advanced just ahead of the tip of the guiding catheter after the ostial lesion has been crossed by a coronary guidewire and stent delivery system. Id. The expanded nitinol legs prevent the entry of the guiding catheter into the target vessel, mark the plane of the aortic wall, and align the tip of the guide with the aorto ostial plane. Id. Such devices include products which travel within a guide catheter to aid in locating the ostium and thus occupy a portion of the limited internal diameter of such catheters.

To locate the true ostium of an artery, a suitable positioning device must create a visual or tactile feedback system that can be utilized during an interventional procedure. Visual and tactile indicators come in many forms. A visual indicator utilizes radio-opaque materials that can be seen under fluoroscopy. These indicators create a reference point for locating the ostial interface. A tactile indicator utilizes a feature that transfers a tactile feel to the proximal end of the device to inform the surgeon, who may be located one meter away from the lesion, that the device is in the proper position. But accurate tactile feedback requires unimpeded, smooth axial movement of such features along and within the length of a catheter that may have a small internal diameter.

The typical guide catheter for delivering, retrieving or re-positioning stents is made of a reinforced tubular polymer. The guide catheter comes in various French sizes. Typically, these sizes range between 6, 7, and 8 French. The French catheter scale is commonly used to measure the outer diameter of cylindrical catheters. http://en.wikipedia.org/wiki/Frenchcatheterscale. The diameter in millimeters of the catheter equals the French size divided by 3. For example, if the French size is 9, the diameter is 3 mm. Id.

As used herein, the term “catheter” means a tube that can be inserted into a body cavity, duct or vessel. http://en.wikipedia.org/wiki/catheter. Catheters allow drainage, injection of fluids or access by surgical devices or instruments. The process of inserting a catheter is called “catheterization”. In most uses, a catheter is a thin, flexible tube. But in some uses, it may be a larger solid tube, for example, to facilitate placement into a particular part of the body. Various catheter tips or guide wires can be used to guide the catheter into the target vessel.

A guide catheter is a conduit for introducing interventional devices during an endovascular procedure. The proximal (extracorporeal) end of the guide catheter may incorporate a luer lock. Conventionally, a luer lock is a system of small-scale fluid fittings that is used for making leak-free connections between a male-taper fitting and its mating female part on medical instruments. http://en.wikipedia.org/wiki/LuerTaper. Luer lock fittings are securely joined by a tabbed hub on the female fitting which screws into threads in a sleeve on the male fitting. Id. Luer components are manufactured from metal or plastic and are available from many companies worldwide. Id. “Luer-LOK” is a registered trademark of Becton Dickinson. In the literature and in this patent application, a “Luer-Lok” style connector is generically referred to as a “luer-lock connector”.

Thus, the luer lock is the conduit that allows a guide catheter to interface with external devices to introduce wires, balloons, stents, contrast media, and/or prevent the back flow of blood out of the guide catheter. The distal (intracorporeal) end of the guide catheter is typically formed into a shape to help the introduction of devices into the vasculature. The shape is formed during the manufacturing of the catheter. Differently shaped catheters are used in different vascular applications.

During a typical procedure, the guide catheter is engaged into the artery to be stented. When engaged, the distal end of the guide catheter is located within the ostial interface. Due to the proximity of the distal end of guide catheter to the ostial interface, it would be desirable to use the distal end of the guide catheter itself to position an ostial locator device.

Illustrative of the prior art are U.S. Pat. Nos. 5,749,890; 6,458,151; 6,659,981; and U.S. Publication Nos. 2004/0181272, 2007/0225788, 2007/0225790, 2007/0239252, 2008/0082155 and 2008/0228146.

One consequence of having an ostial locating device inside the catheter, as described in U.S. Publication Nos. 2007/0225790 and 2008/0082155 is that the effective inside diameter of the catheter is reduced. As a result, less space is available without interference for use by other devices, such as stents.

For example, a typical inside diameter of a catheter may be 1/80,000 inches. A locating device having a wall thickness of 1/5,000 inches may be used, which when doubled leaves an effective amount of useful space of 1/70,000 inches. This may make it difficult to manipulate uninterruptedly and without interference a stent that may be suitable for use in a patient's legs, which may other things being equal, require a larger stent. Simply stated, the stent needs room to maneuver within the internal diameter of the catheter and locating device. Such clearance issues affect the feel that is transmitted to the physician.

One adverse consequence of such arrangements is that a stent may become damaged. In some cases, the stent may be fragile and easily bent by unwanted interference with a surrounding structure. If bent, fracture may result. Stent fracture may not be apparent during an operational procedure. Indeed, it may not be evident until sometime later when the stent exhibits fatigue fracture upon repeated exposure to blood flow that may pulsate, for example, every two seconds. Another adverse consequence is that a ruptured stent may pierce a balloon that is used in angioplasty, or cause the balloon to inflate non-uniformly.

In light of such observations, it would be desirable to have an ostial locating device that is located on the outside of an internal lumen and beyond the end of the catheter so that there is less risk of interference between the inside lumen of the catheter and a medical device such as a stent that passes therethrough.

### SUMMARY

The inventive catheter system solves ostial-aorta positioning problems by creating an assembly that precisely and predictably locates the ostium of an artery, preferably using a guide catheter.

To assist in placing a medical device or a medication proximate an ostium of an artery to treat an ostial lesion repeatably, safely and economically, the catheter system disclosed herein optionally has a guide catheter with a tubular wall and an internal lumen with a proximal end and a distal end. Outside the guide catheter is a sheath that also has a proximal end and a distal end. Inside the sheath is an internal lumen along virtually the entire length of which the guide catheter slidably and twistably extends. Once in place, in one embodiment, the guide catheter can be stiffened to avoid prolapse.

An actuating mechanism is associated with the proximal end of the sheath, the guide catheter or the sheath and guide catheter.

To facilitate twisting, and thus flexibility during insertion into a tortuous vasculature in one embodiment, rounded connecting components are disposed within axially extending rounded tunnels defined in the tubular wall of the guide catheter. Thus, the guide catheter and the connecting components extend slidably axially within the sheath. Relatedly, the connecting components can twist in relation to their associated tunnels during insertion of the catheter system into the vasculature for compliance therewith. Each connecting component also has a proximal end that extends from the actuating mechanism.

An ostial locating member is attached to and extends from the distal ends of at least some of the connecting components. The ostial locating member lies within the sheath before deployment and after the ostial locating member is retracted.

Preferably, the ostial locating member, like a tripod, has three flexible, radially and axially extending struts that each terminate distally in radio-opaque feet. When deployed, the struts are positioned transversely in relation to a longitudinal axis of the guide catheter. After placement, the struts sit astride an ostium defined by a branch vessel and lie in an ostial plane after being advanced against the ostium by distally directed axial sliding movement of the connecting components. In one embodiment, the axial sliding movement of the connecting components is initiated by the actuating mechanism.

In use, the radio-opaque feet and the struts protrude beyond the distal end of the sheath when the radio-opaque feet of the ostial locating member are positioned astride the ostium.

In each embodiment, deployment of the ostial locating member is performed by the actuating mechanism that is provided extracorporeally, proximal to the surgeon. This mechanism may take many forms, such as linear, helical or rotational forms, but for simplification, a linear mechanism is shown in all embodiments.

One recommended procedure for placing a medical device such as a stent proximate ostial lesion involves mounting the locating device at the distal ends of connecting components that run along tunnels defined in the tubular wall of the catheter. The locating device in one embodiment has flexible distal, radially and outwardly extending struts that are advanced to a deployed position that is ahead of the distal end of the guiding catheter. After the struts have been deployed, they prevent further incursion of the guiding catheter into the vessel into which the stent is to be placed or medicine is to be administered. When deployed, the struts define the plane of the aortic wall. In that position, the struts help align the distal end of the guiding catheter with the ostial plane. A stent or other device can then be predictably and accurately placed in the ostial lesion by a controlled emergence from the catheter tip, unimpeded by the locating member.

### BRIEF DESCRIPTION OF THE DRAWINGS

FIG. 1 is a schematic sectional view of a distal region of a sheath, a guiding catheter and an ostial locating mechanism that extends from connecting components; the locating mechanism has radially extending struts that sit astride a vessel that is stenosed;

FIG. 2 is a side view of a catheter assembly according to one embodiment thereof;

FIG. 3 is a cross sectional view in the plane F-F in FIG. 2;

FIG. 4 is a cross sectional view in the plane G-G in FIG. 2;

FIG. 5 is a cross sectional view in the plane E-E in FIG. 2;

FIG. 6 is a cross sectional view in the plane D-D in FIG. 2;

FIG. 7 illustrates one form of an ostial locating device in a deployed configuration situated at the distal end of connecting members that extend in one embodiment along tunnels in the wall of the catheter;

FIG. 8 is a sectional view of an embodiment of the guide catheter in which the catheter wall houses tunnels along which connecting members in the form of fibers slidably extend;

FIG. 9 is a side view of the embodiment shown in FIG. 8.

### DETAILED DESCRIPTION OF THE PREFERRED EMBODIMENT

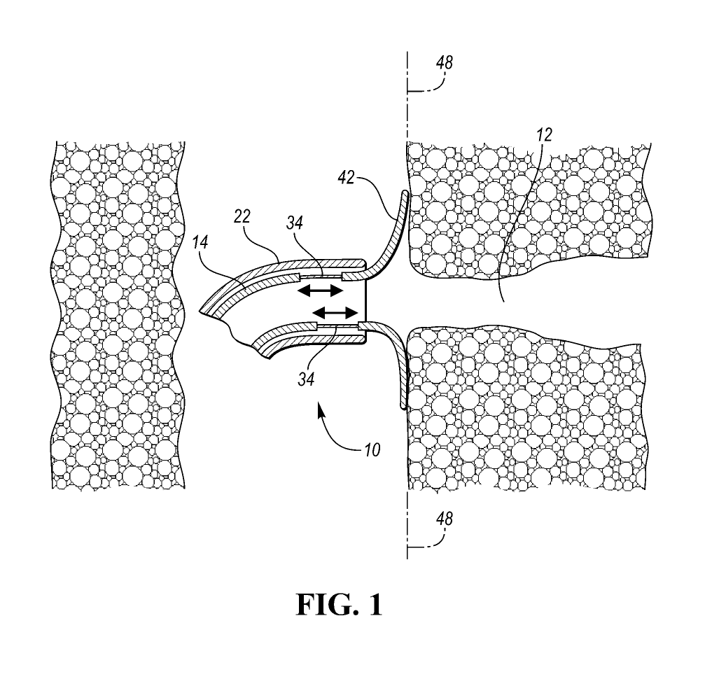

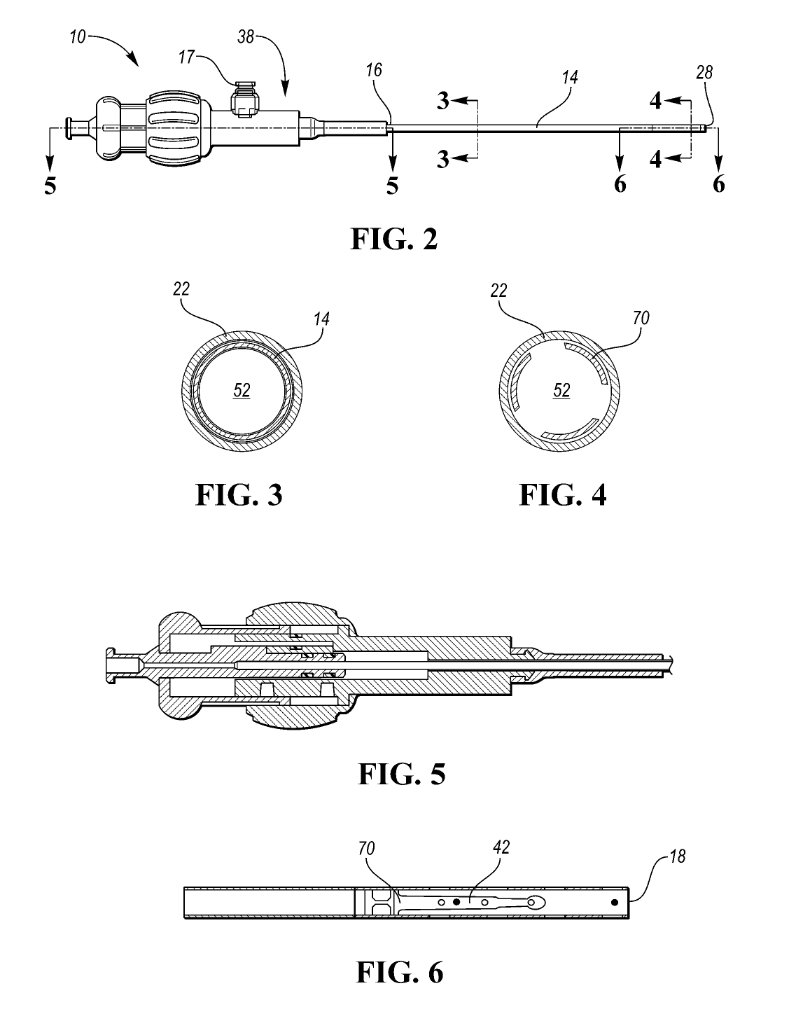

FIGS. 1-6 conceptually represent a catheter system 10 including a guide catheter 14 that has a proximal end 16 which interfaces via an actuating mechanism 38 with a surgeon. Conventionally, external devices are optionally connected through a luer-lock or comparable arrangement. The luer-lock is attached to tubing associated with the guide catheter 14. A proximal hub may incorporate actuation components that interface with an ostial locating member 42. FIGS. 2 & 5 illustrate other conventional parts of the actuating mechanism 38. They include a female flush connector, a torque ball, an advancement module, an advancement knob, an O-ring, an adhesive, and a gripping surface.

Further distally, guide catheter 14 in one embodiment can be considered as reinforced tubing with a single lumen 52. Typically, the tubing 14 has an external hydrophilic coating or laminate to improve lubricity and is available in a variety of lengths.

FIGS. 3-4 respectively show an intermediate section and a section G-G (FIG. 2). FIG. 4 illustrates a distal end region of the sheath 22 and a portion of the struts 70. If desired the sheath 22 or the catheter 14 may include a liner etched with PTFE or a similar material. A laminate such as Pebax 5533 may also be deployed. If desired an optional encapsulated braid may be included.

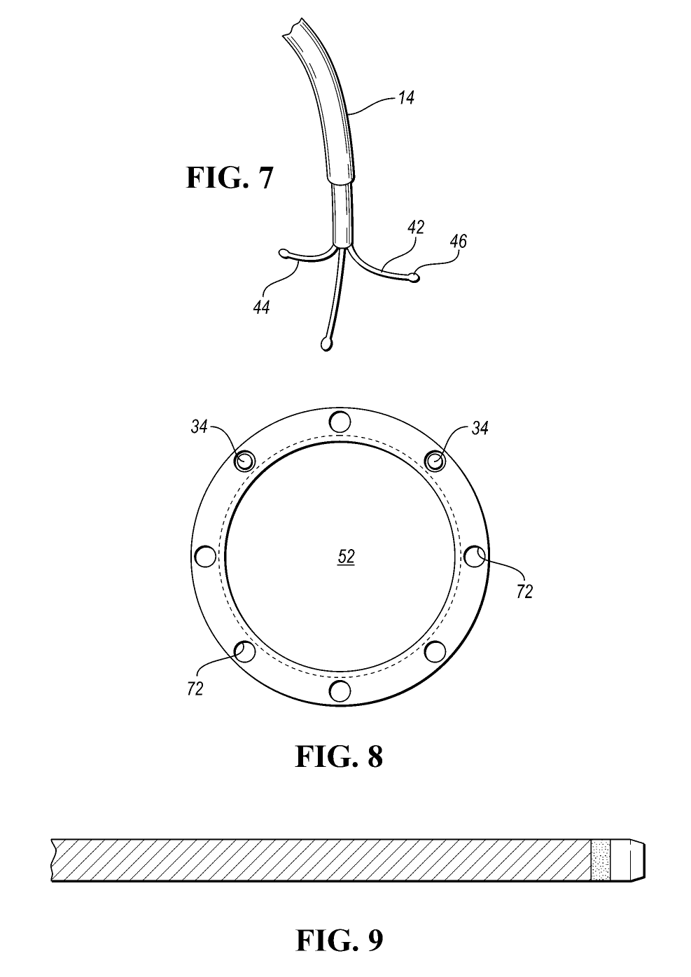

Referring to FIG. 7, it will be appreciated that the guide catheter 14 may be provided with variously shaped distal ends that support an ostial locating member 42. If desired, a locating tip bonding ring (FIG. 9) may facilitate joinder between the connecting components and the ostial locating members.

Continuing with primary reference to FIGS. 1-6, the distal end 18 of the guide catheter 14 interfaces with arteries. Typically, it is soft and non-traumatic to prevent arterial damage.

In the description of various alternative inventive embodiments, it will be appreciated that the luer-lock or comparable subsystem will preferably be incorporated into proximal assembly associated with the linear actuator 38. A feature that is also common to the alternate inventive embodiments is the guide catheter 14, including a covering sheath 22 or tubing. In the catheter system described and depicted, the sheath 22 preferably has a thin wall. In FIG. 8, the connecting components 34 are depicted as wire or fiber-like structures that have a rounded, preferably circular cross-section. Thus, they can readily slide and twist within the tunnels 32 formed in the catheter wall.

Actuation of the ostial locating members 42 is enabled by the linear or axial sliding of one or more connecting components 34.

In one embodiment, distal legs are formed from tubing to create a multi-pronged locating member 42. Preferably, like a tripod, this member has three struts. Although other techniques could be used, one way to prepare the distal legs 70 is by a laser cutting operation. The distal legs 70 can move relative to the catheter 14.

The proximal end of the distal legs 70 is fixedly attached to one or more connecting components 34 which extend back to the linear actuator 38. The entire distal leg component 70 can move in relation to a wall of the catheter 14.

When the linear actuator 38 is moved to the open position, the distal legs 70 slide axially. When moved to the completely open position, the distal legs 70 extend beyond the distal end 18 of the catheter 14. When extended, the multi-pronged structure forms a landing surface. The material for the distal legs 70 is preferably a shape memory material (e.g., nitinol).

When the distal legs 70 are in the open position, they can interface with ostium of the vessel, and be used to locate the ostial interface.

Optionally, the locating members 42 have struts or petals 70 with an upwardly turned end 46 to facilitate their placement against the vessel wall without injury.

FIG. 8 depicts tunnels 32 defined within a wall of the catheter, through which the connecting components 34 may move. Optionally, the catheter 14 may be stiffened by axially extending fibers. In one embodiment the fibers run outside a closed coil 70 (FIGS. 8 & 9). The fibers are free to slide and are connected to the distal legs 70. The actuating mechanism 38 includes a handle or button to lock the fibers by applying tension, thus stiffening the catheter 14.

Once the flexible catheter 14 is tracked into position, the connecting components 34 are locked to minimize any additional flexing that would be required for a prolapse to occur.

In some embodiments, the braid may take the form of a “Chinese Handcuff” which can be stiffened under tension. One form of stiffenable device is disclosed in commonly owned U.S. Pat. No. 8,663,196 which is incorporated herein by reference.

### Experiments

One set of experiments included an evaluation of ostial locating guide catheters. The study was designed to evaluate in vivo safety and effectiveness of a first generation prototype of the device disclosed herein.

It was noted that the device includes a guide catheter for improving the accuracy of stent placement at the ostium of a blood vessel, at such locations as the aortic/coronary junction. One objective of the device was to provide greater long-term success of stents placed in patients with coronary artery disease. As contemplated, the device will reliably assist with the accurate placement of stents and significantly decrease mortality in stent placement procedures, together with decreasing surgical time and cost.

The subject device was compared to a commercially available counterpart. Functional grading included an evaluation of tracking performance and the ability to reach target locations for each device.

In one experiment a sheath was advanced in the vasculature under fluoroscopic guidance over a 0.035″ guidewire and secured with a suture.

Both catheters were introduced through the sheath over the guidewire.

As to a first generation prototype of the device disclosed herein, it was observed that (1) introduction was smooth with no unnecessary blood loss during placement; (2) shaft strength was excellent; (3) the first generation catheter was difficult to land properly due to improper shape and inadequate tip flexibility; (4) struts would often proceed down the ostium; (5) the struts did not fully retract, which can cause complications such as catching on a stent. As to a commercially available catheter, in comparison, it was observed that (1) there was excessive bleeding upon device insertion; (2) it was difficult to insert as the device “kinks out” the guidewire (prolapses); (3) it was difficult to deploy and retain deployment; (4) it did not deploy stents.

Here are the reference numerals and respective design components:

| Reference No. | Feature |
|---|---|
| 10 | Catheter assembly |
| 12 | Ostial lesion |
| 14 | Guide catheter |
| 16 | Proximal end |
| 17 | External devices |
| 18 | Distal end |
| 19 | Luer-lock |
| 22 | Sheath |
| 34 | Connecting component |
| 38 | Actuating mechanism |
| 42 | Ostial locating member |
| 44 | Struts |
| 46 | Feet |
| 48 | Ostial plane |
| 70 | Coil |
| 72 | Tunnel |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A catheter system for placing a medical device proximate an ostium to treat an ostial lesion, the catheter system comprising:
a guide catheter having a tubular wall, and an uninterrupted internal lumen through which the medical device is placed without interference, the guide catheter also having with a proximal end and a distal end;
a sheath that has a proximal end, a distal end, and an internal lumen along virtually the entire length of which the guide catheter slidably extends;
an actuating mechanism associated with the proximal end of the sheath, the guide catheter or the sheath and guide catheter;
rounded connecting components disposed within axially extending rounded tunnels defined in the tubular wall of the guide catheter, the guide catheter and the connecting components extending slidably axially within the sheath and the connecting components being able to twist in relation to their associated tunnels during insertion of the catheter system into a tortuous vasculature for compliance therewith, at least some of the connecting components having a proximal end that extends from the actuating mechanism; and
an ostial locating member that is attached to and extends from the distal ends of at least some of the connecting components so that the ostial locating member lies within the sheath before deployment and when the ostial locating member is retracted;
the ostial locating member having flexible, radially and axially extending struts that each terminate distally in outwardly extending radio-opaque feet which are positioned transversely in relation to a longitudinal axis of the guide catheter, sit astride an ostium defined by a branch vessel and lie in an ostial plane when they are advanced against the ostium by distally directed axial sliding movement of the connecting components in relation to the guide catheter, the axial sliding movement of the connecting components being initiated by the actuating mechanism;
the radio-opaque feet and the struts protruding beyond the distal end of the sheath when the radio-opaque feet of the ostial locating member are positioned astride the ostium.

2. The catheter system of claim 1 where the diameter of the inner lumen of the guide catheter is between 6 Fr and 8 Fr and the diameter of the outside of the sheath is between 7.5 Fr and 9.5 Fr.

3. The catheter system of claim 1, wherein the actuating mechanism is biased to a forward position, so that upon release of the actuating mechanism, the feet of the ostial locating member extend from the distal end of the sheath and guide catheter and flare outwardly, thereby presenting a surface area that creates a relatively soft landing site to the ostium without injury to an arterial wall.

4. The catheter system of claim 1 wherein the ostial locating member includes Nitinol.

5. The catheter system of claim 1 wherein the ostial locating member is formed from a shape memory alloy.

6. The catheter system of claim 1 wherein the actuating mechanism includes a knob that moves longitudinally and helically or rotationally so that an axial force can be applied distally outwardly and proximally inwardly to the connecting components.

7. The catheter system of claim 1, wherein each of the connecting components slide within tunnels that lie within the tubular wall of the guide catheter.

8. The catheter system of claim 1, wherein the struts when extended have an outside diameter that lies between 4 and 15 mm.

9. The catheter system of claim 1 wherein the guide catheter may be stiffened after the struts are deployed at the ostium by a tensioning force exerted on at least one connecting member to avoid prolapse.

10. The catheter system of claim 1, wherein the actuating mechanism is biased to a forward position, so that upon release, the ostial locating member opens.

11. The catheter system of claim 1, wherein the ostial locating member has radially expandable legs.

12. The catheter system of claim 1 wherein the actuating mechanism includes a knob by which longitudinal or rotational or combined longitudinal and rotational force can be applied to one or more connecting components by which the connecting components can be translated axially.

13. A method for placing a medical device proximate an ostium of an artery or for delivering a medicine to treat an ostial lesion, comprising the steps of:
providing a catheter system having a guide catheter with a tubular wall, a sheath that extends around the guide catheter, the sheath having an internal lumen through which the guide catheter slidably extends, the guide catheter also having an internal lumen through which the medical device is placed or medicine is delivered at the ostium without interruption along the internal lumen of the guide catheter;
locating an actuating mechanism at the proximal end of the guide catheter;
disposing rounded connecting components within the tubular wall of the guide catheter, the connecting components twistingly and slidingly axially within and in relation to the tubular wall, the guide catheter being slidable axially inside the sheath;
mounting an ostial locating member with struts terminating in radio-opaque feet at the distal ends of the connecting components; and
deploying the ostial locating member and its struts by movement of the actuating mechanism, thereby advancing the ostial locating member toward the ostium so that the radio-opaque feet lie in or near the ostial plane.
